(19) United States
(12) Patent Application Publication
(10) Pub. No.: US 2020/0090790 A1
(43) Pub. Date: Mar. 19, 2020

(54) SYSTEM AND METHOD FOR MANAGING CLINICAL TRIALS DATA

(71) Applicant: Innoplexus AG, Eschborn (DE)

(72) Inventors: Gaurav Tripathi, Pune (IN); Jaimin Mehta, Baner (IN); Dileep Dharma, Pune (IN); Vatsal Agarwal, Rampur (IN); Tapashi Mandal, Baruipur (IN); Esha Pandita, Aligarh (IN); Snehal Wagh, Ahemednagar (IN)

(21) Appl. No.: 16/366,218

(22) Filed: Mar. 27, 2019

(30) Foreign Application Priority Data
Mar. 27, 2018 (GB) 1804882.7

(51) Int. Cl. G16H 10/20; G06K 9/62; G06F 16/901

(52) U.S. Cl. CPC G16H 10/20; G06F 16/901; G06K 9/6215; G06K 9/628

(57) ABSTRACT

A system and method for managing clinical trials data. The system includes a database arrangement operable to store existing data sources and aggregated clinical trial; and a processing module communicably coupled to the database arrangement. The processing module operable to identify a set of clinical trials; extract clinical trials data from existing data sources; classify the clinical trial entries into one or more predefined classes; compare the clinical trial entries in each of the one or more predefined classes, to identify similarity or dissimilarity between the clinical trial entries in a predefined class; compile the first and second aggregated clinical trial entries to obtain class-specific clinical trial entries corresponding to each of the one or more predefined classes; and collate class-specific clinical trial entries corresponding to each of the one or more predefined classes to obtain an aggregated clinical trial.

### CROSS-REFERENCE TO RELATED APPLICATIONS

This application claims the benefit under 35 U.S.C. § 119(a) and 37 CFR § 1.55 to UK Patent Application No. GB1804882.7, filed on Mar. 27, 2018, the entire content of which is incorporated herein by reference.

### TECHNICAL FIELD

The present disclosure relates generally to data processing; and more specifically, to processing of pharmaceutical data. Furthermore, the present disclosure relates to systems that manages clinical trials data. Moreover, the present disclosure relates to methods for management of clinical trials data. Moreover, the present disclosure also relates to computer readable medium containing program instructions for execution on a computer system, which when executed by a computer, cause the computer to perform method steps for managing clinical trials data.

### BACKGROUND

Typically, whenever a new drug is to be launched for sale to the public, a proof is required to establish that the drug is safe for use and effective in treating some condition. In order to validate this, drug companies carry out experiments and test of the drug. The experiments and tests conducted may comprise giving drug to subjects (namely, humans and animals) in some specific composition and ratio. Furthermore, results of such experiments and tests are provided to an approving body in form of clinical trials in order to authenticate the experiment. Moreover, the clinical trials are conducted in varying proportions of constituents, different environmental conditions, for different diseases and so forth. Consequently, each drug may have a plurality of clinical trials associated therewith in different countries, at different point of times, for different diseases and so forth. Additionally, the plurality of clinical trials may be needed by a researcher experimenting on the drug, a patient who wants to use the drug for condition and the like.

In order to access such plurality of clinical trials a user may need to visit each of the approving body having a clinical trial associated with the drug. Furthermore, such a method of accessing the plurality of clinical trials may be time consuming and require manual effort by the user. Additionally, such plurality of clinical trials may include enormous amount of clinical trial data that may be redundant as well as unmanageable. Consequently, the user may be vulnerable to miss out on some useful information and experience a lot of difficulty in order to analyze the plurality of clinical trials.

Therefore, in light of the foregoing discussion, there exists a need to overcome the aforementioned drawbacks associated with management of clinical trials data.

### SUMMARY

The present disclosure seeks to provide a system that manages clinical trials data. The present disclosure also seeks to provide a method of managing clinical trials data. The present disclosure also seeks to provide a computer readable medium, containing program instructions for execution on a computer system, which when executed by a computer, cause the computer to perform method steps for managing clinical trials data. The present disclosure seeks to provide a solution to the existing problem of time and labor consuming task of analysing plurality of clinical trials. An aim of the present disclosure is to provide a solution that overcomes at least partially the problems encountered in the prior art, and provides an effortless, simple and less time-consuming method of managing clinical trials data.

In one aspect, an embodiment of the present disclosure provides system that manages clinical trials data, wherein the system includes a computer system, wherein the system comprises:
a database arrangement operable to store existing data sources and aggregated clinical trial; and
a processing module communicably coupled to the database arrangement, the processing module operable to:
   identify a set of clinical trials, wherein the set of clinical trials comprises clinical trials having a relation therebetween;
   extract clinical trials data from existing data sources, wherein clinical trials data comprises clinical trial entries of each of the clinical trials in the set of clinical trials;
   classify the clinical trial entries into one or more predefined classes;
   compare the clinical trial entries in each of the one or more predefined classes, to identify similarity or dissimilarity between the clinical trial entries in a predefined class,
      wherein upon identification of similarity between clinical trial entries in the predefined class, one of the similar clinical trial entries is stored in a first aggregated clinical trial entry corresponding to the predefined class; and
      wherein upon identification of dissimilarity between clinical trial entries in the predefined class, the dissimilar clinical trial entries are stored in a second aggregated clinical trial entry corresponding to the predefined class;
   compile the first and second aggregated clinical trial entries to obtain class-specific clinical trial entries corresponding to each of the one or more predefined classes; and
   collate class-specific clinical trial entries corresponding to each of the one or more predefined classes to obtain an aggregated clinical trial.

In another aspect, an embodiment of the present disclosure provides a method of managing clinical trials data, wherein the method includes using a computer system, wherein the method comprises:
identifying a set of clinical trials, wherein the set of clinical trials comprises clinical trials having a relation therebetween;
extracting clinical trials data from existing data sources, wherein clinical trials data comprises clinical trial entries of each of the clinical trials in the set of clinical trials;
classifying the clinical trial entries into one or more predefined classes;
comparing the clinical trial entries in each of the one or more predefined classes, to identify similarity or dissimilarity between the clinical trial entries in a predefined class,
   wherein upon identification of similarity between clinical trial entries in the predefined class, one of the similar clinical trial entries is stored in a first aggregated clinical trial entry corresponding to the predefined class; and
   wherein upon identification of dissimilarity between clinical trial entries in the predefined class, the dissimilar clinical trial entries are stored in a second aggregated clinical trial entry corresponding to the predefined class;
compiling the first and second aggregated clinical trial entries to obtain class-specific clinical trial entries corresponding to each of the one or more predefined classes; and
collating class-specific clinical trial entries corresponding to each of the one or more predefined classes to obtain an aggregated clinical trial.

In yet another aspect, an embodiment of the present disclosure provides a computer readable medium, containing program instructions for execution on a computer system, which when executed by a computer, cause the computer to perform method steps for managing clinical trials data, the method comprising the steps of:
identifying a set of clinical trials, wherein the set of clinical trials comprises clinical trials having a relation therebetween;
extracting clinical trials data from existing data sources, wherein clinical trials data comprises clinical trial entries of each of the clinical trials in the set of clinical trials;
classifying the clinical trial entries into one or more predefined classes;
comparing the clinical trial entries in each of the one or more predefined classes, to identify similarity or dissimilarity between the clinical trial entries in a predefined class,
   wherein upon identification of similarity between clinical trial entries in the predefined class, one of the similar clinical trial entries is stored in a first aggregated clinical trial entry corresponding to the predefined class; and
   wherein upon identification of dissimilarity between clinical trial entries in the predefined class, the dissimilar clinical trial entries are stored in a second aggregated clinical trial entry corresponding to the predefined class;
compiling the first and second aggregated clinical trial entries to obtain class-specific clinical trial entries corresponding to each of the one or more predefined classes; and
collating class-specific clinical trial entries corresponding to each of the one or more predefined classes to obtain an aggregated clinical trial.

Embodiments of the present disclosure substantially eliminate or at least partially address the aforementioned problems in the prior art, and enables an effective and optimal way of managing clinical trials data.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 illustrates steps of a method of managing clinical trials data, in accordance with an embodiment of the present disclosure; and

FIG. 2 is a block diagram of a system that manages clinical trials data, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

In overview, embodiments of the present disclosure are concerned with aggregation of clinical trials data and specifically to, providing an aggregated set of data for a plurality of clinical trials.

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, an embodiment of the present disclosure provides a system that manages clinical trials data, wherein the system includes a computer system, wherein the system comprises:
a database arrangement operable to store existing data sources and aggregated clinical trial; and
a processing module communicably coupled to the database arrangement, the processing module operable to:
   identify a set of clinical trials, wherein the set of clinical trials comprises clinical trials having a relation therebetween;
   extract clinical trials data from existing data sources, wherein clinical trials data comprises clinical trial entries of each of the clinical trials in the set of clinical trials;
   classify the clinical trial entries into one or more predefined classes;
   compare the clinical trial entries in each of the one or more predefined classes, to identify similarity or dissimilarity between the clinical trial entries in a predefined class,
      wherein upon identification of similarity between clinical trial entries in the predefined class, one of the similar clinical trial entries is stored in a first aggregated clinical trial entry corresponding to the predefined class; and
      wherein upon identification of dissimilarity between clinical trial entries in the predefined class, the dissimilar clinical trial entries are stored in a second aggregated clinical trial entry corresponding to the predefined class;
   compile the first and second aggregated clinical trial entries to obtain class-specific clinical trial entries corresponding to each of the one or more predefined classes; and
   collate class-specific clinical trial entries corresponding to each of the one or more predefined classes to obtain an aggregated clinical trial.

In another aspect, an embodiment of the present disclosure provides a method of managing clinical trials data, wherein the method includes using a computer system, wherein the method comprises:
identifying a set of clinical trials, wherein the set of clinical trials comprises clinical trials having a relation therebetween;
extracting clinical trials data from existing data sources, wherein clinical trials data comprises clinical trial entries of each of the clinical trials in the set of clinical trials;
classifying the clinical trial entries into one or more predefined classes;
comparing the clinical trial entries in each of the one or more predefined classes, to identify similarity or dissimilarity between the clinical trial entries in a predefined class,
   wherein upon identification of similarity between clinical trial entries in the predefined class, one of the similar clinical trial entries is stored in a first aggregated clinical trial entry corresponding to the predefined class; and
   wherein upon identification of dissimilarity between clinical trial entries in the predefined class, the dissimilar clinical trial entries are stored in a second aggregated clinical trial entry corresponding to the predefined class;
compiling the first and second aggregated clinical trial entries to obtain class-specific clinical trial entries corresponding to each of the one or more predefined classes; and
collating class-specific clinical trial entries corresponding to each of the one or more predefined classes to obtain an aggregated clinical trial.

The present disclosure provides the aforementioned system for managing clinical trials data and the aforementioned method of managing clinical trials data. The described method allows a collective representation of plurality of clinical trials data. Consequently, a person is provided with an optimal information content regarding a specific drug, condition and so forth. Additionally, the method provides a faster, effortless and less time-consuming way of analysing bulk of data in an organised and structured manner. Moreover, the method enables substantial elimination of data redundancy. Furthermore, the system described herein is simple, reliable and effective.

The computer system relates to at least one computing unit comprising a central storage system, processing units and various peripheral devices. Optionally, the computer system relates to an arrangement of interconnected computing units, wherein each computing unit in the computer system operates independently and may communicate with other external devices and other computing units in the computer system.

The term “system that manages” is used interchangeably with the term “system for managing”, wherever appropriate i.e. whenever one such term is used it also encompasses the other term.

Throughout the present disclosure, the term “clinical trial” relates to a database containing results and other information related to tests, experiments and observations carried out on a subject (for example, humans and animals) in clinical research. Furthermore, such tests, experiments and observations are performed to obtain specific information related to biomedical or behavioural interventions, including new treatments (such as novel vaccines, drugs, dietary choices, dietary supplements and medical devices and so forth) and known interventions that require further study and comparison. Additionally, the clinical trial is carried out in a number of phases involving different constraints applied for conducting the clinical trial. Moreover, the clinical trial may have a number of versions depending upon date of the trial. Furthermore, the clinical trials for a specific drug may be conducted in different geographical locations and under varying environmental conditions. Such clinical trials may be provided to an approving body in order to validate authentication of the clinical trial and approve use thereof by the public. Furthermore, the clinical trials have a relation therebetween based on drug under the clinical trial, geographical location of the clinical trial, applicability of the drug in treating a specific condition and so forth. Furthermore, the clinical trial includes information regarding one or more related clinical trial conducted in different countries and/or at different point in time.

As mentioned previously, the method of managing clinical trials data comprises identifying the set of clinical trials, wherein the set of clinical trials comprises clinical trials having a relation therebetween. Furthermore, a processing module is operable to identify the set of clinical trials, wherein the set of clinical trials comprises clinical trials having a relation therebetween. Specifically, a plurality of clinical trials are identified based on one or more common information stored therein. For example, clinical trials of drugs for use in a specific condition may be related to each other, clinical trials for a specific drug in different countries may have a relation therebetween and so forth. Moreover, a clinical trial includes information associated with clinical trials related thereto. Specifically, the information associated with related clinical trials may comprise trials IDs of the related clinical trials, country of origin of the related clinical trials and so forth. Such information is used to identify the set of clinical trials.

Throughout the present disclosure, the term “processing module” relates to a computational element that is operable to respond to and process instructions for managing clinical trials. Optionally, the processing module includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “processing module” may refer to one or more individual processors, processing devices and various elements associated with a processing device that may be shared by other processing devices. Additionally, the one or more individual processors, processing devices and elements are arranged in various architectures for responding to and processing the instructions that drive the system.

Optionally, the processing module is operable to identify the set of clinical trials from a list of clinical trials. The processing module is operable to access a list of clinical trials and select the set of clinical trials based on one or more constraints such as an alphabetical order, time of the clinical trial, condition to be treated, requirement stated through instructions and so forth. Furthermore, the set of clinical trials is identified by accessing the list of clinical trials sequentially or randomly. Specifically, the processing module may be operable to access the list of clinical trials in a sequential order based on position thereof in the list or the clinical trials may be accessed randomly within the list based on one or more constraints. More optionally, the set of clinical trials is identified manually by a user. The user may select the set of clinical trials by means of a user interface, a drop down menu, and so forth. Furthermore, the user may select a specific clinical trial. Subsequently, the user may identify the related clinical trials from the information included in the specific clinical trials to obtain the set of clinical trials.

In a first example, a clinical trial conducted in United States may include clinical trials data related to a drug used in treating pneumonia, composition of the drug, geographical location of the clinical trial, phase of the clinical trial and so forth. Furthermore, clinical trials carried out for the drug for pneumonia in different geographical locations like India, Australia and China and at different points in time may be related to each other based on the drug for pneumonia. Additionally, the clinical trial conducted in United States for the drug for pneumonia may have information therein regarding one or more clinical trials for the drug for pneumonia conducted in different geographical locations. Furthermore, a clinical trial, for the drug for pneumonia, conducted in year 2004 may be related to different versions thereof namely, clinical trials for the drug for pneumonia, conducted in 2006, 2008, 2014.

Furthermore, the set of clinical trials related to each other are stored in existing data sources. The term “existing data sources” relates to organized or unorganized bodies of digital information regardless of manner in which data is represented therein. Optionally, the existing data sources are structured and/or unstructured. Optionally, the existing data sources may be hardware, software, firmware and/or any combination thereof. For example, the existing data sources may be in form of tables, maps, grids, packets, datagrams, files, documents, lists or in any other form. The existing data sources include any data storage software and systems, such as, for example, a relational database like IBM, DB2, Oracle 9 and so forth. Moreover, the existing data sources may include the data in form of text, audio, video, image and/or a combination thereof. Furthermore, each of the approving bodies may have one or more existing data sources associated thereto for storing clinical trials. Moreover, a database arrangement is operable to store existing data sources.

Throughout the invention, the term ‘database arrangement’ as used herein relates to an organized body of digital information regardless of the manner in which the data or the organized body thereof is represented. Optionally, the database arrangement may be hardware, software, firmware and/or any combination thereof. For example, the organized body of related data may be in the form of a table, a map, a grid, a packet, a datagram, a file, a document, a list or in any other form. The database arrangement includes any data storage software and systems, such as, for example, a relational database like IBM DB2 and Oracle 9. Optionally, the database arrangement may be used interchangeably herein as database management system, as is common in the art. Furthermore, the database management system refers to the software program for creating and managing one or more databases. Optionally, the database arrangement may be operable to supports relational operations, regardless of whether it enforces strict adherence to the relational model, as understood by those of ordinary skill in the art. Additionally, the database arrangement populated by data elements. Furthermore, the data elements may include data records, bits of data, cells, and are used interchangeably herein and all intended to mean information stored in cells of a database. Optionally, the database arrangement may store the existing data sources in distributed or centralized manner. Furthermore, the existing data sources may be used for accessing information associated to the set of clinical trials.

Furthermore, the method of managing clinical trials comprises extracting clinical trials data from existing data sources, wherein clinical trials data comprises clinical trial entries of each of the clinical trials in the set of clinical trials. Specifically, the existing data sources are accessed and information associated to the set of clinical trials is extracted (namely, copied) to form a set of data containing information associated with the set of clinical trials only. Furthermore, the clinical trials entries refer to each of the data stored in the clinical trials. Referring to the first example, clinical trial data for clinical trials related to the drug for treating pneumonia may have clinical trials entries such as drug name, phase, composition, date of clinical trial and so forth. Specifically, the clinical trial entries may be “Levofloxacin” for the drug name, “2” for phase of the clinical trial, date of clinical trial February 2006 to November 2006 and so forth. Beneficially, extraction of the clinical trials data associated to the set of clinical trials reduces the bulk of data to be analysed. Specifically, the processing module is operable to extract clinical trials data from existing data sources. The processing module is configured to access existing data sources and analyse the clinical trials data in order to extract the clinical trials entries associated with the set of clinical trials. Optionally, the extracted clinical trial entries may have an additional field associated therewith, wherein the additional field may denote name of the country where the respective clinical trial has been conducted.

Optionally, the extracting clinical trials data from existing data sources comprises associating a clinical trial identifier with clinical trial entries of each of the clinical trial in the set of clinical trials. Furthermore, the processing module is operable to associate the clinical trial identifier with clinical trial entries of each of the clinical trial in the set of clinical trials. Specifically, the clinical trial identifier may be a clinical trial ID, country name, and so forth for establishing a relation between the clinical trial entries and respective clinical trial thereof. Additionally, each of the clinical trial entries may be associated with clinical trial identifier thereof in order to uniquely identify a specific clinical trial. Furthermore, such association of clinical trial identifier also enables identification of clinical trial data associated with a specific clinical trial among the extracted clinical trials data.

As mentioned previously, the method further comprises classifying the clinical trial entries into one or more predefined classes. Furthermore, the processing module is operable to classify the clinical trial entries into one or more predefined classes. Specifically, each of the clinical trial entries in the set of clinical trials that comprise data related to same field of clinical trial are grouped together. Beneficially, the classifying of the clinical trials entries differentiates the clinical trials entries in the set of the clinical trials. In a second example, a set of clinical trials data may include three clinical trials for “Ethambutol”, a medicine used in treatment of tuberculosis. Additionally, the three clinical trials may be conducted in US, Brazil and Argentina. The set of clinical trials include clinical trials data such as, country, trial ID, condition, phase and so forth. Furthermore, clinical trial entries containing trial ID of each of the clinical trials for “Ethambutol” are grouped together in a predefined class. Similarly, clinical trial entries comprising country of each of the clinical trials are grouped together in one predefined class. Furthermore, clinical trial entries comprising phase of each of the clinical trials are grouped together in another predefined class and clinical trial entries comprising condition associated with each of the clinical trials are grouped together in a predefined class. Additionally, the classes are predefined based on information included in each of the clinical trials.

Moreover, the method further comprises: comparing the clinical trial entries in each of the one or more predefined classes, to identify similarity or dissimilarity between the clinical trial entries in a predefined class. Furthermore, the processing module is operable to compare the clinical trial entries in each of the one or more predefined classes, to identify similarity or dissimilarity between the clinical trial entries in a predefined class. The clinical trial entries in a specific predefined class are analysed with respect to other clinical trial entries in the specific predefined class. Referring to the second example, clinical trial for “Ethambutol” in US may be in Phase 2, clinical trial for “Ethambutol” in Brazil may be in Phase 1 and clinical trial for “Ethambutol” in Argentina may be in Phase 2. Furthermore, phase of each of the clinical trials included in the predefined class are compared with each other. In another example, clinical trial entries in the set of three clinical trials for predefined class “Study Intervention” may be “Paracetamol”, “Paracetamol”, and “Paracetamol and Citrizine”. Therefore, in such example, similarity is identified between the clinical trials entries “Paracetamol” and “Paracetamol” and the clinical trial entry “Paracetamol and Citrizine” is identified as dissimilar clinical trial entry.

Optionally, identification of similarity or dissimilarity between the clinical trial entries in the predefined class is performed by determining a similarity score. Specifically, the processing module is operable to identify similarity or dissimilarity between the clinical trial entries in the predefined class by determining a similarity score. Additionally, a similarity score indicates similarity or dissimilarity among clinical trial entries in the predefined class. Furthermore, a maximum similarity score indicates identical information stored in two or more clinical trial entries. Alternatively, a similarity score less than maximum indicates difference in information stored in two or more clinical trial entries. In an example, drugs names “Alkeran” and “Leukeran” may have a similarity score of 80%, maximum being 100%. Consequently, the drug names are considered to be different. Referring to the second example, similarity score of clinical trial entries comprising phase associated with clinical trial for “Ethambutol” in US and clinical trial for “Ethambutol” in Argentina may have a 100% similarity score. Consequently, the information stored in the clinical trial entries comprising phase associated with clinical trial for “Ethambutol” in US and clinical trial for “Ethambutol” in Argentina are considered as similar. In another implementation, the similarity score may be identified on a scale of zero to one, wherein similarity score of identical clinical trial entries may be identified as one. Additionally, similarity score of clinical entries with a difference therebetween may be identified as zero. Furthermore, the similarity score may be calculated using edit distance technique.

Furthermore, upon identification of similarity between clinical trial entries in the predefined class, one of the similar clinical trial entries is stored in a first aggregated clinical trial entry corresponding to the predefined class. For an instance, when two or more clinical trial entries stored in the predefined class represent similar (namely, identical) information; only one clinical trial entry is retained and remaining clinical trial entries are discarded. Consequently, one of the similar entries is stored in a first aggregated clinical trial entry. Specifically, upon identification of similarity between clinical trial entries in the predefined class, the processing module is operable to store one of the similar clinical trial entries in a first aggregated clinical trial entry corresponding to the predefined class. In an example, clinical trial entries, in a set of six clinical trials, for predefined class: “Study Intervention”, may be “Paracetamol”, “Paracetamol”, “Etuximab”, “Paracetamol and Citrizine”, “Cetuximab” and “Etuximab”. Therefore, in such example, similarity is identified between “Paracetamol” and “Paracetamol”, and “Etuximab” and “Etuximab”. Consequently, the clinical trial entries “Paracetamol” and “Etuximab” are stored in the first aggregated clinical trial entry corresponding to the predefined class: “Study intervention”.

Moreover, upon identification of dissimilarity between clinical trial entries in the predefined class, the dissimilar clinical trial entries are stored in a second aggregated clinical trial entry corresponding to the predefined class. Specifically, upon identification of dissimilarity between clinical trial entries in the predefined class, the processing module is operable to store the dissimilar clinical trial entries in a second aggregated clinical trial entry corresponding to the predefined class. The second aggregated class includes clinical trial entries in the predefined class having dissimilar information. Consequently, the predefined class is associated with the first aggregated clinical trial entry and the second aggregated clinical trial entry comprising similar and dissimilar information respectively, wherein the similar and dissimilar information is obtained from the clinical trial entries included in the predefined class. Referring to the aforementioned example, clinical entries “Paracetamol and Citrizine” and “Cetuximab”, in the set of five clinical trials are stored in the second aggregated clinical trial entry corresponding to the predefined class: “Study Intervention”

Optionally, the identification of similarity or dissimilarity may be calculated by associating a frequency table for each of the information in the clinical trial entries in the predefined class. Referring to the second example, a frequency table may be associated for the predefined class containing clinical trial entries for phase of the clinical trials included in the set of clinical trials. Furthermore, the frequency table may have a frequency of two for clinical trial entry containing phase “2”. Consequently, one of the clinical trial entries containing phase “2” may be stored in a first aggregated clinical trial entry corresponding to the predefined class. Moreover, the frequency table may have a frequency of one for clinical trial entry containing phase “1”. Consequently, the clinical trial entry is considered to have dissimilarity and is stored in a second aggregated clinical trial entry corresponding to the predefined class. Furthermore, each of the information in the clinical trial entry with a frequency of more than one is included in the first aggregated clinical trial entry and each of the information in the clinical trial entry with a frequency of one is included in the second aggregated clinical trial entry.

Furthermore, the method comprises compiling the first and second aggregated clinical trial entries to obtain a class-specific clinical trial entry corresponding to each of the one or more predefined classes. Specifically, the processing module is operable to compile the first and second aggregated clinical trial entries to obtain a class-specific clinical trial entry corresponding to each of the one or more predefined classes. The first aggregated clinical trial entry and the second aggregated clinical trial entry are combined to obtain the class-specific clinical trial entry. Furthermore, the class-specific clinical trial entry contains all the information stored in the predefined class without redundancy. Each of the predefined classes in the set of clinical trials have a class-specific clinical trial corresponding thereto. Beneficially, the class-specific clinical trial enables representation of information in the predefined class without redundancy and information loss. Referring to the aforementioned example, in the set of six clinical trials, first aggregated clinical trial entry and second aggregated clinical trial entry corresponding to the predefined class “Study intervention” are compiled to obtain the class-specific clinical trial entry corresponding to the predefined class “Study intervention”. Moreover, the class-specific clinical trial entry corresponding to the predefined class “Study intervention” comprises the clinical trial entries “Paracetamol”, “Etuximab”, “Paracetamol and Citrizine”, and “Cetuximab”. Similarly, first and second aggregated clinical trial entries are compiled to obtain class-specific clinical trial entries corresponding to each of the one or more predefined classes

Optionally, compiling the first and second aggregated clinical trial entries comprises providing the clinical trial identifier, associated with the clinical trial entries, in the class-specific clinical trial entry. Furthermore, the processing module is operable to provide the clinical trial identifier, associated with the clinical trial entries, in the class-specific clinical trial entry. Specifically, the clinical trial identifier may be a clinical trial ID, country name, inventor Id and so forth. Additionally, each of the class-specific clinical trial entry may be associated with clinical trial identifier thereof in order to uniquely identify a specific clinical trial. Furthermore, such association of clinical trial identifier also enables identification of clinical trial entry associated with a specific clinical trial in the class-specific clinical trial entry.

Furthermore, the method comprises collating class-specific clinical trial entries corresponding to each of the one or more predefined classes to obtain an aggregated clinical trial. Specifically, the processing module is operable to collate class-specific clinical trial entries corresponding to each of the one or more predefined classes to obtain an aggregated clinical trial. Furthermore, the class-specific clinical trial entries corresponding to each of the predefined class are assembled together. Beneficially, such assembling of the class-specific clinical trial entries provides a single document containing clinical trial entries associated with the set of clinical trials. Furthermore, the single document forms the aggregated clinical trial providing a collection of clinical trials data associated with the set of clinical trials. Additionally, the database arrangement is operable to store the aggregated clinical trial. Furthermore, the processing module is operable to access the database arrangement in order to retrieve the aggregated clinical trial. In an example, the aggregated clinical trial may be done in a tabular form, using charts or some other mode of data representation.

Optionally, the clinical trial entries of each of the clinical trials are time stamped. Specifically, the processing module is operable to time stamp the clinical trial entries of each of the clinical trials. Furthermore, the clinical trial entries may be associated with a year of clinical trial. Consequently, the time stamp enables to predict relevance of the clinical trials data associated with the clinical trials. In an example, a clinical trial entry with a time stamp of 2008 may be considered to be more relevant than a clinical trial entry with a time stamp of 1990. More optionally, a relevancy score is determined based on the time stamps of the clinical trial entries, wherein the relevancy score is associated with a version of the clinical trial. Specifically, the processing module is operable to determine a relevancy score based on the time stamps of the clinical trial entries, wherein the relevancy score is associated with a version of the clinical trial. The time stamps of two or more clinical trial entries in the predefined class are compared and the clinical trial entry with a higher value of time stamp is given a higher relevancy. Additionally, the clinical trial entry with a lower value of time stamp is given a lower relevancy. The higher relevancy score may denote most recent version of a clinical trial for a drug conducted in a country. Furthermore, such relevancy score may be included in the aggregated clinical trial in order to indicate most relevant information. In an embodiment, the relevancy score may be higher for the clinical entries with a version showing successful results. Subsequently, when comparing clinical trials entries of different clinical trials, only the clinical trial entries with highest relevancy score may be compared.

In an exemplary implementation of the present disclosure, a set of clinical trials conducted in countries United States (US), Germany and China are identified. Specifically, the set of clinical trials have a relation therebetween. Consequently, clinical trials data related to the set of clinical trials may be extracted. Subsequently, the clinical trials entries are classified in the predefined classes: “Trial ID”, “Condition”, “Drugs”, “Phase”, and “Date”. Specifically, the clinical trials data related to US, Germany and China may comprise clinical trial entries as shown in the charts 1.1, 1.2 and 1.3.

CHART 1.1
US

| Trial ID | Condition | Drugs | Phase | Date |
|---|---|---|---|---|
| US2009 | Atopic | Baricinib, | 1 | February |
| (also published | Dermatitis | Placebo, | | 2016-December |
| as GM4080, | | Triamcinolone | | 2016 |
| CH7409) | | | | |

CHART 1.2
Germany

| Trial ID | Condition | Drugs | Phase | Date |
|---|---|---|---|---|
| GM4080 | Atopic | Baricinib, | 2 | January 2016-February 2016 |
| | Dermatitis | Placebo | | |

CHART 1.3
China

| Trial ID | Condition | Drugs | Phase | Date |
|---|---|---|---|---|
| CH7409 | Atopic | Baricinib, | 2 | March 2015-November 2016 |
| | Dermatitis | Placebo | | |

It is to be understood that the aforementioned charts only include information of clinical trial that is required for the example. Furthermore, a clinical trial may include additional information like compound composition, number of persons enrolled in clinical trial and so forth. Additionally, a clinical trial may not be in presented format and may be presented in any other structure. The charts include exemplary information fields of the clinical trial for treating “Atopic Dermatitis”.

Furthermore, the clinical trials in each of the predefined class are compared to identify a similarity or dissimilarity therebetween. In an example, the clinical trial entries in the class “Condition” are compared and a similarity in identified therebetween. Similarly, in the class “Drug”, the clinical trial entries “Baricinib, Placebo” and “Baricinib, Placebo” are identified as similar clinical trial entries and “Triamcinolone” is identified as the dissimilar clinical trial entry. Consequently, similar clinical trial entries are stored in a first aggregated clinical trial entry and dissimilar clinical trial entries are stored in a second aggregated clinical trial entry corresponding to the predefined class, as shown for the class “Drug” in Chart 1.4.

CHART 2.1

| First aggregated clinical trial | Second aggregated clinical trial |
|---|---|
| entry | entry |
| Baricinib, Placebo | Triamcinolone |

Subsequently, the first aggregated clinical trial entry and the second aggregated clinical trial entry are compiled to obtain a class-specific clinical trial entry corresponding to the predefined class, as shown for the predefined class “Drugs” in chart 3.1.

CHART 3.1
Class-specific clinical trial entry

Baricinib, Placebo, Triamcinolone

It will be appreciated that the aforementioned steps of comparing clinical trial entry in a predefined class, storing in a first aggregated clinical trial entry and a second aggregated clinical trial entry and subsequently compiling the first aggregated clinical trial entry and the second aggregated clinical trial entry to obtain a class-specific clinical trial entry, is executed for each of the predefined classes included in the exemplary clinical trials.

Furthermore, class-specific clinical trial entries corresponding to each of the predefined classes are collated together to form an aggregated clinical trial, as shown in chart 4.1.

CHART 4.1
Aggregated Clinical Trial

| Trial ID | Condition | Drugs | Phase | Date |
|---|---|---|---|---|
| US2009, | Atopic | Baricinib, | 1 | February 2016-December |
| GM4080, | Dermatitis | Placebo, | 2 | 2016 |
| CH7409 | | Triamcinolone | | January 2016-February |
| | | | | 2016 |
| | | | | March 2015-November |
| | | | | 2016 |

Optionally, a clinical trial identifier (such as the geographical location of the clinical trial) may be associated with clinical trial entries in predefined classes such as “Trial ID”, “Phase”, and “Date”.

Furthermore, there is disclosed a computer readable medium, containing program instructions for execution on a computer system, which when executed by a computer, cause the computer to perform method steps for managing clinical trials data. The method comprises steps of identifying a set of clinical trials, wherein the set of clinical trials comprises clinical trials having a relation therebetween; extracting clinical trials data from existing data sources, wherein clinical trials data comprises clinical trial entries of each of the clinical trials in the set of clinical trials; classifying the clinical trial entries into one or more predefined classes; comparing the clinical trial entries in each of the one or more predefined classes, to identify similarity or dissimilarity between the clinical trial entries in a predefined class. Furthermore, upon identification of similarity between clinical trial entries in the predefined class, one of the similar clinical trial entries is stored in a first aggregated clinical trial entry corresponding to the predefined class; and upon identification of dissimilarity between clinical trial entries in the predefined class, the dissimilar clinical trial entries are stored in a second aggregated clinical trial entry corresponding to the predefined class. Subsequently, the method comprises compiling the first and second aggregated clinical trial entries to obtain class-specific clinical trial entries corresponding to each of the one or more predefined classes; and collating class-specific clinical trial entries corresponding to each of the one or more predefined classes to obtain an aggregated clinical trial.

Optionally, the machine-readable non-transient data storage media comprises one of a floppy disk, a hard disk, a high capacity read only memory in the form of an optically read compact disk or CD-ROM, a DVD, a tape, a read only memory (ROM), and a random access memory (RAM).

### DETAILED DESCRIPTION OF THE DRAWINGS

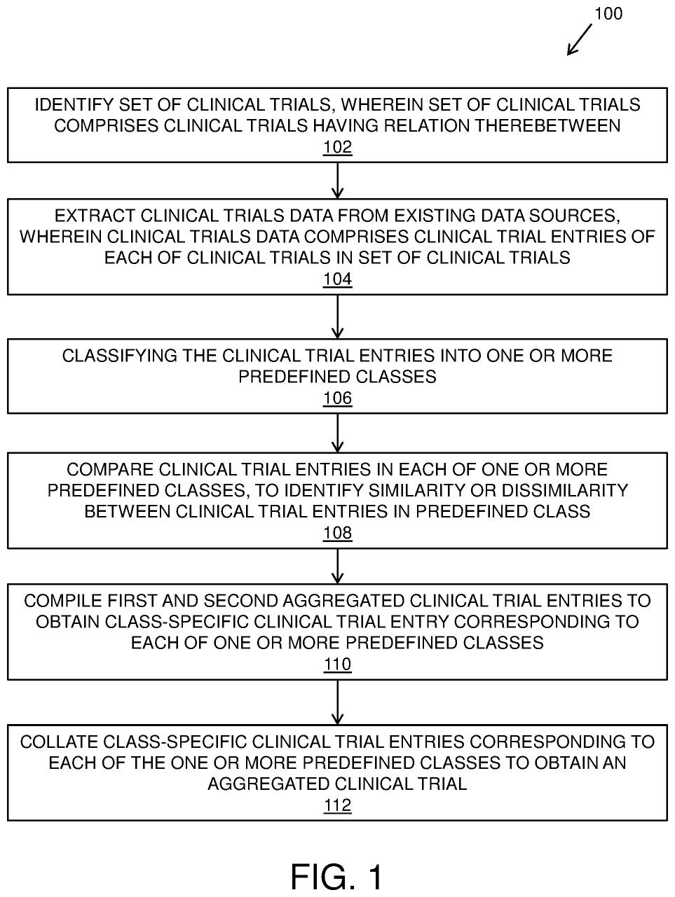

Referring to FIG. 1, illustrated are steps of a method 100 for managing clinical trials data, in accordance with an embodiment of the present disclosure. At a step 102, a set of clinical trials are identified. Additionally, the set of clinical trials comprises clinical trials having a relation therebetween. At a step 104, clinical trials data from existing data sources are extracted. Specifically, the clinical trials data comprises clinical trial entries of each of the clinical trials in the set of clinical trials. At a step 106, the clinical trial entries are classified into one or more predefined classes. At a step 108, the clinical trial entries in each of the one or more predefined classes are compared to identify similarity or dissimilarity between the clinical trial entries in a predefined class. Moreover, upon identification of similarity between clinical trial entries in the predefined class, one of the similar clinical trial entries is stored in a first aggregated clinical trial entry corresponding to the predefined class. Furthermore, upon identification of dissimilarity between clinical trial entries in the predefined class, the dissimilar clinical trial entries are stored in a second aggregated clinical trial entry corresponding to the predefined class. Subsequently, the first and second aggregated clinical trial entries are compiled to obtain a class-specific clinical trial entry corresponding to the predefined class. At a step 110, the first and second aggregated clinical trial entries are compiled to obtain a class-specific clinical trial entry corresponding to each of the one or more predefined classes. At a step 112, class-specific clinical trial entries are collated corresponding to each of the one or more predefined classes to obtain an aggregated clinical trial.

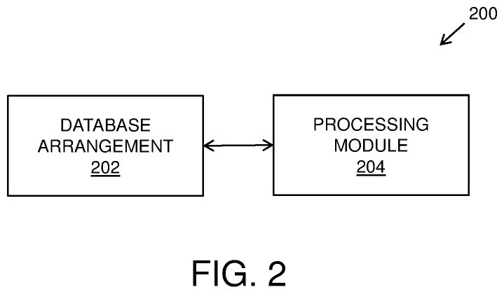

Referring to FIG. 2, illustrated is a block diagram of a system 200 that manages clinical trials data, in accordance with an embodiment of the present disclosure. The system 200 comprises a database arrangement 202 operable to store existing data sources and aggregated clinical trial. Furthermore, the database arrangement is operably coupled to a processing module 204. The processing module 204 is operable to extract a set clinical trials data from the existing data sources.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A system that manages clinical trials data, wherein the system includes a computer system, wherein the system comprises:
a database arrangement operable to store existing data sources and aggregated clinical trial; and
a processing module communicably coupled to the database arrangement, the processing module operable to:
identify a set of clinical trials, wherein the set of clinical trials comprises clinical trials having a relation therebetween;
extract clinical trials data from existing data sources, wherein clinical trials data comprises clinical trial entries of each of the clinical trials in the set of clinical trials;
classify the clinical trial entries into one or more predefined classes;
compare the clinical trial entries in each of the one or more predefined classes, to identify similarity or dissimilarity between the clinical trial entries in a predefined class,
wherein upon identification of similarity between clinical trial entries in the predefined class, one of the similar clinical trial entries is stored in a first aggregated clinical trial entry corresponding to the predefined class; and
wherein upon identification of dissimilarity between clinical trial entries in the predefined class, the dissimilar clinical trial entries are stored in a second aggregated clinical trial entry corresponding to the predefined class;
compile the first and second aggregated clinical trial entries to obtain class-specific clinical trial entries corresponding to each of the one or more predefined classes; and
collate class-specific clinical trial entries corresponding to each of the one or more predefined classes to obtain an aggregated clinical trial.

2. The system of claim 1, wherein a user is operable to identify the set of clinical trials manually.

3. The system of claim 1 wherein the processing module is operable to identify the set of clinical trials from a list of clinical trials.

4. The system of claim 1, wherein the set of clinical trials is identified by accessing the list of clinical trials sequentially or randomly.

5. The system of claim 1, wherein the processing module is operable to associate a clinical trial identifier with clinical trial entries of each of the clinical trial in the set of clinical trials.

6. The system of claim 1, wherein the processing module is operable to provide the clinical trial identifier, associated with the clinical trial entries, in the class-specific clinical trial entry.

7. The system of claim 1, wherein the processing module is operable to identify similarity or dissimilarity between the clinical trial entries in a predefined class by determining a similarity score.

8. The system of claim 1, wherein the processing module is operable to time stamp the clinical trial entries of each of the clinical trials.

9. The system of claim 1, wherein the processing module is operable to determine a relevancy score based on the time stamps of the clinical trial entries, wherein the relevancy score is associated with a version of the clinical trial.

10. A method of managing clinical trials data, wherein the method includes using a computer system, wherein the method comprises:
identifying a set of clinical trials, wherein the set of clinical trials comprises clinical trials having a relation therebetween;
extracting clinical trials data from existing data sources, wherein clinical trials data comprises clinical trial entries of each of the clinical trials in the set of clinical trials;
classifying the clinical trial entries into one or more predefined classes;
comparing the clinical trial entries in each of the one or more predefined classes, to identify similarity or dissimilarity between the clinical trial entries in a predefined class,
wherein upon identification of similarity between clinical trial entries in the predefined class, one of the similar clinical trial entries is stored in a first aggregated clinical trial entry corresponding to the predefined class; and
wherein upon identification of dissimilarity between clinical trial entries in the predefined class, the dissimilar clinical trial entries are stored in a second aggregated clinical trial entry corresponding to the predefined class;
compiling the first and second aggregated clinical trial entries to obtain class-specific clinical trial entries corresponding to each of the one or more predefined classes; and
collating class-specific clinical trial entries corresponding to each of the one or more predefined classes to obtain an aggregated clinical trial.

11. The method of claim 10, wherein extracting clinical trials data from existing data sources comprises associating a clinical trial identifier with clinical trial entries of each of the clinical trial in the set of clinical trials.

12. The method of claim 10, wherein compiling the first and second aggregated clinical trial entries comprises providing the clinical trial identifier, associated with the clinical trial entries, in the class-specific clinical trial entry.

13. The method of claim 10, wherein identification of similarity or dissimilarity between the clinical trial entries in a predefined class is performed by determining a similarity score.

14. The method of claim 10, wherein the clinical trial entries of each of the clinical trials are time stamped.

15. The method of claim 10, wherein a relevancy score is determined based on the time stamps of the clinical trial entries, wherein the relevancy score is associated with a version of the clinical trial.

16. A computer readable medium, containing program instructions for execution on a computer system, which when executed by a computer, cause the computer to perform method steps for managing clinical trials data, the method comprising the steps of:
identifying a set of clinical trials, wherein the set of clinical trials comprises clinical trials having a relation therebetween;
extracting clinical trials data from existing data sources, wherein clinical trials data comprises clinical trial entries of each of the clinical trials in the set of clinical trials;
classifying the clinical trial entries into one or more predefined classes;
comparing the clinical trial entries in each of the one or more predefined classes, to identify similarity or dissimilarity between the clinical trial entries in a predefined class,
wherein upon identification of similarity between clinical trial entries in the predefined class, one of the similar clinical trial entries is stored in a first aggregated clinical trial entry corresponding to the predefined class; and
wherein upon identification of dissimilarity between clinical trial entries in the predefined class, the dissimilar clinical trial entries are stored in a second aggregated clinical trial entry corresponding to the predefined class;
compiling the first and second aggregated clinical trial entries to obtain class-specific clinical trial entries corresponding to each of the one or more predefined classes; and
collating class-specific clinical trial entries corresponding to each of the one or more predefined classes to obtain an aggregated clinical trial.
